(19) United States
(12) Patent Application Publication
(10) Pub. No.: US 2002/0028985 A1
(43) Pub. Date: Mar. 7, 2002

(54) Fluorescent endoscope apparatus

(75) Inventors: Kazuhiro Tsujita, Kaisei-machi (JP); Kazuo Hakamata, Kaisei-machi (JP)

Correspondence Address: SUGHRUE, MION, ZINN, MACPEAK & SEAS, PLLC, 2100 Pennsylvania Avenue, N.W., Washington, DC, 20037-3202, US

(73) Assignee: FUJI PHOTO FILM CO., LTD.

(21) Appl. No.: 09/946,464

(22) Filed: Sep. 6, 2001

(30) Foreign Application Priority Data
Sep. 7, 2000 (JP) 271324/2000

(51) Int. Cl. A61B 1/06

(52) U.S. Cl. 600/160; 600/178

(57) ABSTRACT

A fluorescent endoscope apparatus that displays as an image the autofluorescent light emitted from a target subject upon the irradiation thereof with an excitation light, wherein the scope means is improved and the size of the apparatus is made compact. An image obtaining unit, an illumination unit, and an excitation/reference-light unit are mounted adjacently on a rack located at a position remote from a scope means. The image obtaining unit, the illumination unit, and the excitation/reference-light unit are connected to the scope means by an image fiber, an illuminating-light guide, and an excitation/reference-light guide, respectively, Further, by making the length of the image fiber and the excitation/reference-light light guide longer than that of the illuminating-light light guide, the range in which the scope means is can be moved is limited only by the illuminating-light light guide.

### BACKGROUND OF THE INVENTION

1. Field of the Invention

The present invention relates in general to a fluorescent endoscope apparatus for displaying as an image the fluorescent light emitted from a target subject upon the irradiation thereof with an excitation light, and in particular to a fluorescent endoscope apparatus in which the scope means to be inserted into the body of a patient is improved and the size of the apparatus is made compact.

2. Description of the Related Art

In the field of fluorescent endoscope apparatuses, technology has been proposed that make use of the fact that the intensity of the fluorescent light emitted from a tissue in the normal state and the intensity of the fluorescent light emitted from a tissue in a diseased state upon the irradiation of a target subject with an excitation light having a wavelength within the wavelength range of the intrinsic fluorophores of the target subject differ. According to fluorescent endoscope apparatuses employing such technology, a target subject is irradiated with an excitation light of a predetermined wavelength range, and by receiving the fluorescent light emitted from the intrinsic fluorophores thereof, the location and range of penetration of a disease can be displayed as an image.

Generally, when a target subject is irradiated with an excitation light, a high-intensity fluorescent light is emitted from the tissue thereof that is in the normal state, as shown by the solid line in FIG. 7, and a low-intensity fluorescent light is emitted from a tissue thereof that is in a diseased state, as shown by the broken line in FIG. 7; therefore, by measuring the intensity of the fluorescent light emitted from a target subject upon the irradiation thereof by an excitation light, it becomes possible to determine if the tissue of the target subject is in the normal or a diseased state.

However, for cases in which the intensity of the fluorescent light emitted from a target subject upon the irradiation thereof by an excitation light is to be displayed as an image, because the surface of a target subject is uneven, the intensity of the excitation light irradiating the surface of the target subject is not uniform. Further, although the intensity of the fluorescent light emitted from a target subject is substantially proportional to the intensity of the excitation light, the intensity of the excitation light decreases in inverse proportion to the square of the distance between the distal end of the excitation light source and the target subject. Therefore, there are cases in which a higher intensity fluorescent light is received from a tissue that is in a diseased state and that is located closer to the distal end of the excitation light source than a tissue that is in the normal state, and the tissue state of the target subject can not be accurately distinguished based solely on the data relating to the intensity of the fluorescent light emitted from the target subject. The applicants of the present application, in order to reduce the precariousness resulting from the problem described above, propose a method of displaying a computed image based on the factor obtained by dividing the ratio of two types of fluorescent light intensities, each of which has been obtained of a different frequency range. That is to say, propose a method of displaying an image based on the difference in the form of the fluorescent spectra reflecting the tissue state of a target subject, a method of displaying a computed image based on the factor obtained by detecting the intensity of the reflected-light reflected from a target subject upon the irradiation thereof with a near-infrared light that exhibits almost uniform absorption characteristics across a wide variety of tissue types, and dividing the detection value obtained thereby by the ratio of the intensity of the fluorescent light; that is, a method of obtaining a value reflecting the yield of fluorescent light, and displaying an image based thereupon.

Further, a fluorescent endoscope apparatus according to the technology described above basically comprises: a scope means that is to be inserted into the body of a patient; an illuminating means; a light source unit for emitting an excitation light and a reference-light or an illuminating-light and an excitation light; and an image obtaining unit for obtaining a reflectance image formed of the reflected-light reflected from a target subject upon the irradiation thereof with an illuminating-light, a fluorescent-light image formed of the fluorescent light emitted from the target subject upon the irradiation thereof by an excitation light, and a reference-light image formed of the reflected-light reflected from the target subject upon the irradiation thereof by a reference-light, or a reflectance image and a fluorescent-light image. Further, according to the configuration of a conventional fluorescent endoscope apparatus, the scope means and the image obtaining unit are directly connected; therefore, it is necessary that the image obtaining unit or the light source unit be activated to operate the directly connected scope means for diagnosis.

However, for cases in which a scope means such as that described above is directly connected to the image obtaining unit, because the scope means and the image obtaining unit must operate together, the weight of the unit becomes heavy and the operability poor. Further, in order to preserve a certain level of operability, one portion thereof must be fixed, whereby a support member and counterweight are required for said fixing, and a problem arises in that the size of the apparatus becomes cumbersome.

### SUMMARY OF THE INVENTION

The present invention has been developed based on consideration of the circumstances described above, and it is a primary objective of the present invention to provide a fluorescent endoscope apparatus in which the operability thereof is improved and the size of the apparatus is made compact.

The first fluorescent endoscope apparatus according to the present invention comprises: an illuminating-light emitting means for emitting an illuminating-light; an excitation light emitting means for emitting an excitation light; a reference-light emitting means for emitting a reference-light; an illuminating-light guiding means for guiding the illuminating-light emitted from the illuminating-light emitting means to the target subject and projecting said illuminating-light onto the target subject; an excitation light guiding means for guiding the excitation light emitted from the excitation light emitting means to the target subject and projecting said excitation light onto the target subject; a reference-light guiding means for guiding the reference-light emitted from the reference-light emitting means to the target subject and projecting said illuminating-light onto the target subject; an light guiding means for guiding a reflectance image formed of the reflected-light reflected from the target subject upon the irradiation thereof by the illuminating-light, a fluorescent-light image formed of the fluorescent light emitted from the target subject upon the irradiation thereof by the excitation light, and a reference-light image formed of the reflected-light reflected from the target subject upon the irradiation thereof by the reference-light; an image obtaining means for obtaining a reflectance image, a fluorescent-light image, and a reference-light image guided to thereto by the light guiding means; and a scope means to be inserted into the body of a patient and which has disposed in the interior thereof the illuminating-light guiding means, the excitation light guiding means, the reference-light guiding means, and the light guiding means; wherein, the image obtaining means is provided at a location remote from the scope means, and the image obtaining means and the light guiding means disposed within the scope means are optically connected by an image obtaining connecting means of a predetermined length.

Here, “an image obtaining connecting means of a predetermined length” refers to a fiber optical cable or the like, capable of guiding aforementioned reflectance image, excitation light image, and reference-light image; “a predetermined length” refers to a length ensuring an adequate range of operability for the scope means. Here, operability refers to the broadness of the range in which the scope means can be moved.

Further, the apparatus can also be of a configuration wherein at least one from among the illuminating-light emitting means, the excitation light emitting means, the reference-light emitting means is provided at a position remote from the scope means, and said at least one light emitting means provided at a position remote from the scope means and the light guiding means corresponding thereto are optically connected by one or more connecting means of a predetermined length.

Here, “connecting means” refers to a light guide, etc. for optically connecting the at least one light emitting means provided at a position remote from the scope means to the light guiding means disposed within the scope means; however, the “connecting means” can be integral with the light guiding means disposed within the scope means.

Further, one connecting means from among aforementioned one or more connecting means and the image obtaining connecting means is of a length determining the distance between the light emitting means or the image obtaining means corresponding to said one connecting means and the scope means, and the connecting means other than said one connecting means having the length determining said distance can be longer in length than said one connecting means determining said distance.

Here, “a length determining the distance” refers to the length that defines the limits of the operability range of the scope means. Accordingly, the connecting means other than said one connecting means defining said distance have substantially no effect on the operability range of the scope means.

Further, the apparatus can be of a configuration wherein at least two from among the illuminating-light emitting means, the excitation light emitting means, the reference-light emitting means, and the image obtaining means provided at a position remote from the scope means are disposed adjacent to one another.

Here, “disposed adjacent to one another” refers to, for a case in which the at least two from among the illuminating-light emitting means, the excitation light emitting means, the reference-light emitting means, and the image obtaining means are provided as separate units, for example, arranging each unit adjacent to one another. In this case, the order in which each unit is arranged is not important; as long as the units are arranged adjacent to one another, any number of arrangements are possible. Further, it is not necessarily required that the aforementioned at least two from among the illuminating-light emitting means, the excitation light emitting means, the reference-light emitting means, and the image obtaining means are provided as separate units; for a case in which at least two from among the illuminating-light emitting means, the excitation light emitting means, the reference-light emitting means, and the image obtaining means are provide within the same unit, the terminals for the connecting means of each means can be adjacent to each other.

The second fluorescent endoscope apparatus according to the present invention comprises: an illuminating-light emitting means for emitting an illuminating-light; an excitation light emitting means for emitting an excitation light; an illuminating-light guiding means for guiding the illuminating-light emitted from the illuminating-light emitting means to the target subject and projecting said illuminating-light onto the target subject; an excitation light guiding means for guiding the excitation light emitted from the excitation light emitting means to the target subject and projecting said excitation light onto the target subject; a light guiding means for guiding a reflectance image formed of the reflected-light reflected from the target subject upon the irradiation thereof by the illuminating-light, and a fluorescent-light image formed of the fluorescent light emitted from the target subject upon the irradiation thereof by the excitation light; an image obtaining means for obtaining a reflectance image and a fluorescent-light image guided thereto by the light guiding means; and a scope means to be inserted into the body of a patient and which has disposed in the interior thereof the illuminating-light guiding means, the excitation light guiding means, and the light guiding means; wherein, the image obtaining means is provided at a location remote from the scope means, and the image obtaining means and the light guiding means disposed within the scope means are optically connected by an image obtaining connecting means of a predetermined length.

Here, “an image obtaining connecting means of a predetermined length” refers to a fiber optical cable or the like capable of guiding aforementioned reflectance image and excitation light image.

Further, at least one from among the illuminating-light emitting means and the excitation light emitting means is provided at a position remote from the scope means, and said at least one light emitting means provided at a position remote from the scope means and the light guiding means corresponding thereto are optically connected by one or more connecting means of a predetermined length.

Still further, one connecting means from among aforementioned one or more connecting means and the image obtaining connecting means is of a length determining the distance between the light emitting means or the image obtaining means corresponding to said one connecting means and the scope means, and the connecting means other than said one connecting means having the length determining said distance can be longer in length than said one connecting means determining said distance.

In addition, the apparatus can be of a configuration wherein at least two from among the illuminating-light emitting means, the excitation light emitting means and the image obtaining means are disposed adjacent to one another other.

Here, “disposed adjacent to one another” refers to, for a case in which the at least two from among the illuminating-light emitting means, the excitation light emitting means, and the image obtaining means are provided as separate units, for example, arranging each unit adjacent to one another. In this case, the order in which each unit is arranged is not important; as long as the units are arranged adjacent to one another, any number of arrangements are possible. Further, it is not necessarily required that the aforementioned at least two from among the illuminating-light emitting means, the excitation light emitting means, and the image obtaining means are provided as separate units; for a case in which at least two from among the illuminating-light emitting means, the excitation light emitting means, and the image obtaining means are provide within the same unit, the terminals for the connecting means of each means can be adjacent to each other.

Note that as to each of the means other than those described above, the explanation thereof is the same as occurred in the first embodiment.

Further, according to the first and second fluorescent endoscope apparatuses of the present invention, the excitation light source can be a GaN laser for outputting light having a wavelength in the 400 to 420 nm range.

According to the fluorescent endoscope apparatus of the present invention: the image obtaining means is provided at a position remote from the scope means; the light guiding means within the image obtaining means and the scope means are optically connected by an image obtaining connecting means; and because a configuration has been adopted so that the load of the image obtaining means is not brought to bear on the scope means, the operability of the scope means can be improved. Further, because it becomes unnecessary to provide the support member and counterweight required to support a conventional scope means, the size of the apparatus can be made compact.

Further, although according to the first fluorescent endoscope of the present invention at least one from among the illuminating-light emitting means, the excitation light emitting means, and the reference-light emitting means is provided at a position remote from the scope means, and according to the second fluorescent endoscope of the present invention at least one from among the illuminating-light emitting means, and the excitation light emitting means is provided at a position remote from the scope means, for cases in which both said at least one light emitting means provided at a position remote from the scope means and the light guiding means corresponding to said light emitting means are optically connected by one or more connecting means of a predetermined length, because the load on the scope means can be further reduced, the operability thereof can be further improved.

Still further, one connecting means from among aforementioned one or more connecting means and the image obtaining connecting means is of a length determining the distance between the light emitting means or the image obtaining means corresponding to said one connecting means and the scope means, and for cases in which the connecting means other than said one connecting means having the length determining said distance are longer in length than said one connecting means determining said distance, only said one connecting means determining said distance limits of the range of operability of the scope means, and because the other connecting means do not limit the range of operability of the scope means, the operability thereof can be further improved.

Additionally, for cases in which at least two from among the illuminating-light emitting means, the excitation light emitting means, the reference-light emitting means and the image obtaining means are disposed adjacent to one another other, or for cases in which at least two from among the illuminating-light emitting means, the excitation light emitting means, and the image obtaining means are disposed adjacent to one another other, according to the first and second fluorescent endoscope apparatus of the present invention, respectively, the operability is improved without unnecessarily increasing the length of the connecting means, and the size of the apparatus can be made compact.

Further, according to the first and second fluorescent endoscopes of the present invention, if a GaN semiconductor laser that emits light having a wavelength in the 400-420 nm wavelength range is employed as the excitation light source, the cost and size of the apparatus can be reduced, and the efficiency of the yield of fluorescent light can be increased.

### BRIEF DESCRIPTION OF THE DRAWINGS

FIG. 1 is a schematic drawing of an embodiment of a fluorescent endoscope apparatus according to the present invention,

FIG. 2 is a side view of the fluorescent endoscope apparatus shown in FIG. 1,

FIG. 3 is a detailed schematic drawing of the scope means and each connecting means employed in the fluorescent endoscope apparatus shown in FIGS. 1 and 2,.

FIG. 4 is a detailed schematic drawing of the endoscope insertion portion of the scope means of the fluorescent endoscope shown in FIGS. 1 and 2,

FIG. 5 is a detailed schematic drawing of the endoscope insertion portion of the scope means of another embodiment of a fluorescent endoscope apparatus according to the present invention,

FIG. 6 is a detailed schematic drawing of the scope means and each connecting means of another embodiment of a fluorescent endoscope apparatus according to the present invention, and

FIG. 7 is a graph showing the distributions of the intensity of the fluorescent light emitted from a tissue in the normal state and from a tissue in a diseased state.

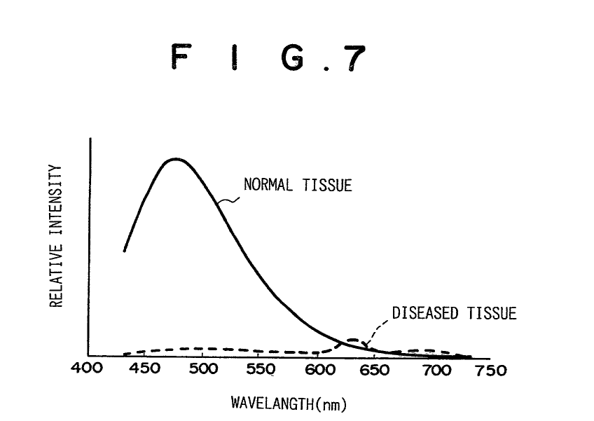

### DESCRIPTION OF THE PREFERRED EMBODIMENTS

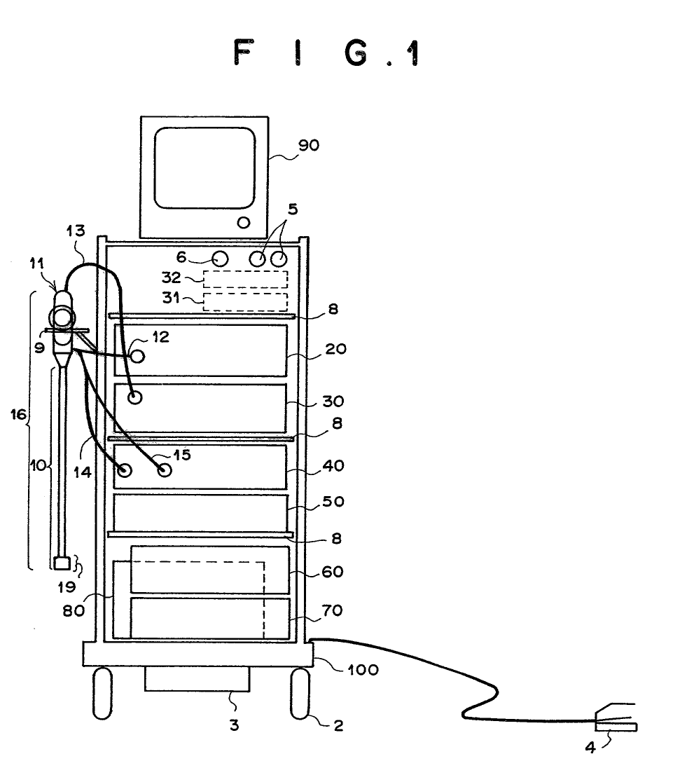

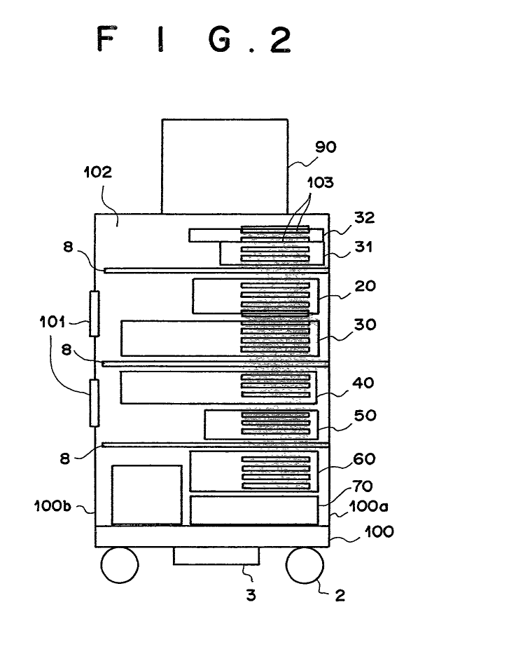

Hereinafter the preferred embodiments of the present invention will be described with reference to the attached drawings. FIG. 1 shows a schematic drawing of an embodiment of a fluorescent endoscope apparatus according to the present invention, and FIG. 2 shows a side view of the fluorescent endoscope apparatus shown in FIG. 1.

The fluorescent light endoscope apparatus according to the current embodiment comprises mainly: a scope means 16 having an endoscope insertion portion 10 to be inserted into the body of a patient; an illuminating-light guide 12, which extends from the scope portion 16, for guiding an illuminating-light; combined excitation light/reference-light light guides 14 and 15 for guiding an excitation light and a reference-light, and an image guide 13; an illumination unit 20 to which the illuminating-light guide 12 is connected; an excitation light/reference-light unit 40 to which the light guides 14 and 15 are connected; an image obtaining unit 30, to which the image guide 13 is connected and, for obtaining an image of a target subject guided thereto by the image guide 13; a monitor 90 for displaying as a visible-image an image obtained by the image obtaining means 30; and a rack 100 onto which the illumination unit 20, the excitation light/reference-light unit 40, the image obtaining unit 30, and the monitor 90 are stacked together.

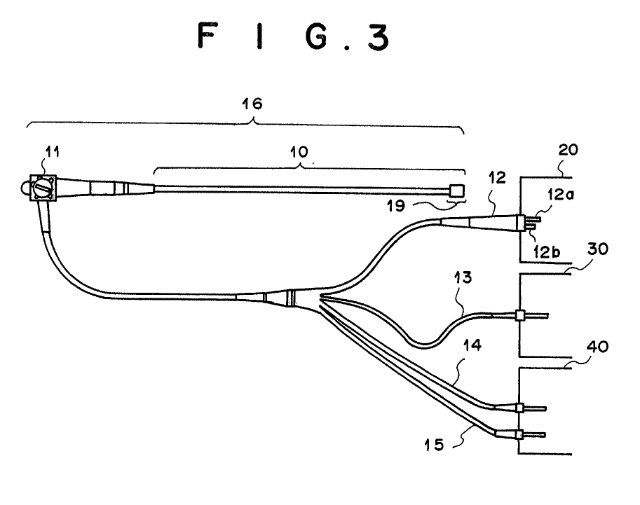

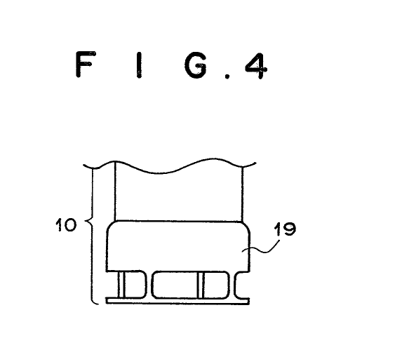

The details of the scope means 16 are shown in the FIGS. 3 and 4. The scope means 16 comprises an endoscope insertion portion 10 to be inserted into the body of a patient, and an operations portion 11 connected to the back end thereof for controlling the operation, etc. of the endoscope insertion portion. One end of the illuminating-light guide 12, the other end of which is connected to the illuminating-light unit 20, one end of the combined excitation light/reference-light light guides 14 and 15, the other end of which is connected to the excitation light/reference-light unit 40 of the operations portion 11, and one end of the image guide 13, the other end of which is connected to the image obtaining means 30, are grouped together and connected to the operations portion 11.

Here, as to the lengths of the illuminating-light guide 12, the combined excitation light/reference-light light guides 14 and 15, and the image guide 13 connected to the scope means 16 of the operations portion 11, the length of the excitation light/reference-light combined-use light guides 14 and 15, and the image guide 13 are longer than that of the illuminating-light guide 12. Accordingly, the distance between the scope means 16 and each unit is determined by the length of the illuminating-light guide 12, and the range of movement of the scope means is limited only by the length of the illuminating-light guide 12. On the other hand, because, as described above, the lengths of the combined excitation light/reference-light light guides 14 and 15, and the length of the image guide 13 is longer than that of the illuminating light guide 12, they have do not limit the range of movement of the scope means 16.

Further, the illuminating-light unit 20 is provided with an air/water transport unit (not shown) for transporting air/water to the target subject through the scope means 16, and the illuminating-light guide 12 comprises an air/water transport line 12b, which is connected to said air/water transport unit, for transporting air and water to the target subject, and a fiber 12a for guiding the illuminating-light. Accordingly, the illuminating-light guide 12 is of a larger diameter than the excitation light/reference-light combined-use light guide 14 and 15, and the image guide 13. Therefore, because said illuminating-light guide 12 is shorter than the excitation light/reference-light combined-use light guides 14 and 15 and the image guide 13, and limits the range of movement of the scope means as well as bears the bulk of the load of the scope means 16 and, as described above, it is preferable that said illuminating-light guide 12 is made to be of a large diameter by combining the air/water transport line or the like as in the configuration described above. Note that according to the current embodiment, although the length of the illuminating-light guide 12 is shorter than that of the combined excitation light/reference-light light guides 14 and 15 and that of the image guide 13, the current embodiment is not limited to being of such configuration; any one from among the other guides, that is, the combined excitation light/reference-light light guides 14 and 15, and the image fiber 13 can be made to be the short guide. Further, in this case, it is desirable that the diameter of the guide that is to be the short guide, as described above, be large.

Further still, the illuminating-light guide 12, the excitation light/reference-light combined-use light guides 14 and 15 and the image guide 13 extend within the endoscope insertion portion 10 to the distal end thereof. The illuminating-light guide 12 is formed of a composite glass fiber, and the excitation light/reference-light combined-use light guides 14 and 15 and the image guide 13 are formed of fused quartz glass fiber.

In addition, as shown in detail in FIG. 3, a cap 19 is installed on the distal end of the endoscope insertion portion 10. The cap 19 is provided as a laser safety measure to prevent injury being caused to a target subject when the excitation light emitting end of the endoscope insertion portion draws too close to the target subject. The cap 19 is structured having an opening on either side thereof so as to prevent reflection of the illuminating-light, the excitation light and the reference-light light emitted from the distal end of the endoscope insertion portion 10.

The illuminating unit 20 is provided with a white-light source (not shown) for emitting white-light, and the excitation/reference-light unit 40 is provided with a GaN semiconductor laser or emitting an excitation light and a reference-light source for emitting a reference-light.

The image obtaining unit 30 is provided with a high-intensity fluorescent-light image obtaining element, a reflectance image obtaining element, and a reference-light image obtaining element for obtaining an autofluorescent-light image, a reflectance image, and a reflected-light image, respectively, and which are connected to the image guide 13, through which said images are conveyed.

It is desirable that the illuminating-light guide 12, the combined excitation light/reference-light light guides 14 and 15 and the image obtaining unit 30 are disposed adjacent to one another as shown in FIGS. 1 and 2. Further, so long as each of the above-described units are disposed adjacent to one another, the order in which said units are arranged does not matter; for example, the illuminating-light unit 20 can be provided between the image obtaining unit 30 and the excitation light/reference-light emitting means 40.

Further, aside from each of the units described above: an image processing PC 70 for subjecting the image data of a fluorescent-light image and a reflectance image obtained by the image obtaining portion 30 to image processing; a video signal processor 50 for converting the image data that has been subjected to image processing to a video signal and outputting said video signal; a semiconductor-laser power supply 60 connected to the excitation/reference-light unit 40; an uninterruptible power supply 80 for supplying power during a blackout; a CCU (Camera Control Unit) 31 mainly for controlling the image obtaining unit 30 and a control unit 32 for controlling the series of operations of each unit, and a power lamp 5 for indicating whether the power supply to the apparatus is turned ON or OFF; an emergency shut-down switch 6 for shutting down the apparatus in an emergency; an insulated transformer 3 for supplying a stable power supply voltage to the apparatus; and a fixing portion 9 for fixing the scope means 16 in place, are provided on the rack 100. The control unit 32 is connected to a foot switch 4 for switching between a reflectance image displaying mode, in which a reflectance image is obtained and displayed, and a composite-image displaying mode, in which a composite-image is obtained and displayed. Note that the power lamp 5 and the emergency shut-down switch 6 are provided on the front panel (not shown) installed on the front face portion 101a of the apparatus. Further, the front panel is provided with openings where the illuminating-light unit 20, the excitation light/reference-light unit 40, the image obtaining means 30, and the video signal processor 50 are installed.

Still further, wheels 2 are provided on the lower portion of the rack 100 so as to provide for ready mobility thereof. Further, the interior of the rack 100 is divided by shelves 8, which have been slideably mounted to provide for improved efficiency when maintenance is to be performed.

In addition, as shown in FIG. 2, a fan 101 is provided on the back portion of the rack 100 for lowering the temperature of the interior space of the rack 100. Further, the side panels 102 of the rack 100 are provided with a plurality of ventilation holes 103 enabling the intake of air therethrough.

Next, the operation of the fluorescent endoscope apparatus according to the current embodiment described above will be described. First, the scope means 16 is removed from the fixing portion 9 of the rack 100, and the endoscope insertion portion 10 is inserted into the body of a patient. Here, the range of movement of the scope means 16 is limited only by the length of the illuminating-lightguide 12; the range of movement of the scope means 16 is not limited by the combined excitation light/reference-light light guides 14 and 15 or the image guide 13.

The GaN semiconductor-laser power supply 50 is activated based on a signal from the control unit 32, and the excitation light is emitted from the GaN semiconductor laser of the excitation light/reference-light unit 40. The excitation light is guided to the distal end of the endoscope insertion portion 10 by both the combined excitation light/reference-light light guides 14 and 15, and is projected therefrom onto the target subject. The autofluorescent-light image formed of the autofluorescent-light emitted from the target subject upon the irradiation thereof by the excitation light enters the input face on the distal end of the image guide 13, is guided therethrough to the image obtaining unit 30, obtained by the high-sensitivity fluorescent-light image obtaining element within the image obtaining unit 30, and the image signal obtained by the high-sensitivity fluorescent-light image obtaining element is output to the image processing PC 70.

Further, the reference-light is caused to be emitted from the reference-light source contained within the excitation light/reference-light unit 40 based upon a signal from the control unit 32. The reference-light is guided to the distal end of the endoscope insertion portion 10 by both the combined excitation light/reference-light light guides 14 and 15, and is projected therefrom onto the target subject. The reference-light image formed of the reflected-light reflected from the target subject upon the irradiation thereof by the reference-light enters the input face on the distal end of the image guide 13, is guided therethrough to the image obtaining unit 30, obtained by the high-sensitivity fluorescent-light image obtaining element within the image obtaining unit 30, and the image signal obtained by reference-light image obtaining element is output to the image processing PC 70.

Note that although according to the current embodiment, the excitation light and the reference-light have each been emitted at different timings, the current embodiment is not limited thereto; both the excitation light and the reference-light can be emitted at the same time.

The image data based on an autofluorescent image that is input to the image processing PC 70 is subjected to a predetermined computational processing, after which color data is assigned thereto and an image signal having color data is formed. Further, a brightness data is assigned to each pixel value of the image data based on the reference-light image, and an image signal having brightness data is formed. The image signal having color data, which is based on the autofluorescent-light image, and the image signal having brightness data, which is based on the reference-light image are combined to form a composite-image signal. After being subjected to another predetermined computational processing, said composite-image signal is output to the video signal processor 50. After converting the image signal that has been subjected to image processing to a video signal, the video signal processor 50 outputs said video signal to the monitor 90. The monitor 90 displays the video signal input thereto as a composite-image.

The series of operations relating to the displaying of a composite-image ad the displaying of a reflectance image described above are controlled by the control unit 32, and the operation of the image obtaining unit 30 is mainly controlled by the CCU 31.

Further, switching between the composite-image displaying mode and the reflectance image displaying mode is performed by depressing the footswitch 4.

Still further, the uninterruptible power supply 80 serves to prevent the resetting of the image processing PC 70 due to a momentary stoppage occurring when the power supply of the apparatus, which is a commercially available power supply, is stopped, as well as to provide temporary power. The insulated transformer 3 serves to stabilize the voltage supplied to the apparatus by the commercially available power source. The power lamp 5 indicates whether the power supply is ON or OFF, and the emergency shutdown switch shuts down the apparatus upon the depression thereof when confronted with an emergency situation requiring that the apparatus be shut down immediately. Note that because it is possible that the endoscope insertion portion is inside the body of a patient when the apparatus has been shut down due to the emergency shutdown switch 6 having been depressed, it is desirable that the apparatus switches to the reflectance image displaying mode thereupon.

Even further still, for cases in which each unit is loaded onto the same rack as shown in FIGS. 1 and 2, the illuminating-light source, etc. generate substantial quantities of heat and the temperature of the interior space of the rack rises, and noise is caused to increase thereby in the image obtaining unit 30 and the CCU 31. In an image obtained by a system for obtaining an image of a weak fluorescent light, the noise due to said heat appears as background noise of a magnitude too large to be ignored, and the S/N ratio of the image deteriorates. In order to control such a rise in temperature, air is drawn in through the ventilation holes 103 provided on the front panel 101a and the side panels 102 of the rack 100, and air is expelled from the back portion 101b of the rack to the exterior of the rack by the fan 101. When this type of method of expelling exhaust air from the back portion of the rack has been adopted, because the heat dissipating means of each unit is at the back thereof, the efficiency of the heat dissipation can be further improved. Further, there is also an effect whereby the heated exhaust air the operator and patient would become exposed to if the exhaust air were to be expelled from the front panel 101a is prevented.

Additionally, the interior of the rack is divided by 8 shelves, which can be slid toward the front when maintenance or the like is to be performed, and each unit can be slid out from the interior of the rack on the respective shelf thereof.

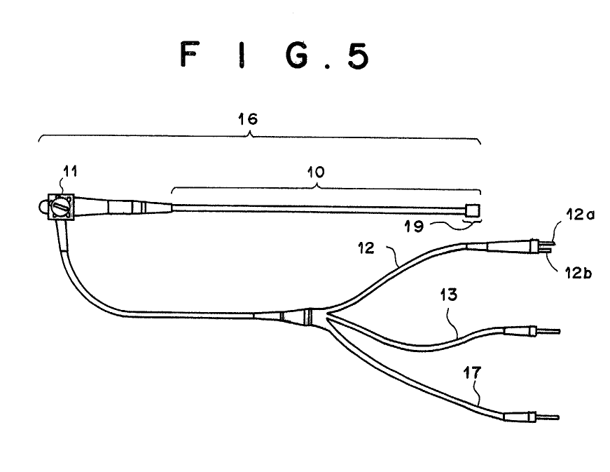

Further, it is not necessarily required that the excitation light/reference-light guide consist of two fibers, a configuration in which said excitation light/reference-light guide consists of a single fiber, such as the configuration shown in FIG. 5 can be adopted. Further, for cases in which the white-light emitted from the illuminating-light unit is jointly used as the reference-light, the excitation light/reference-light guide 17 can be an excitation light guide.

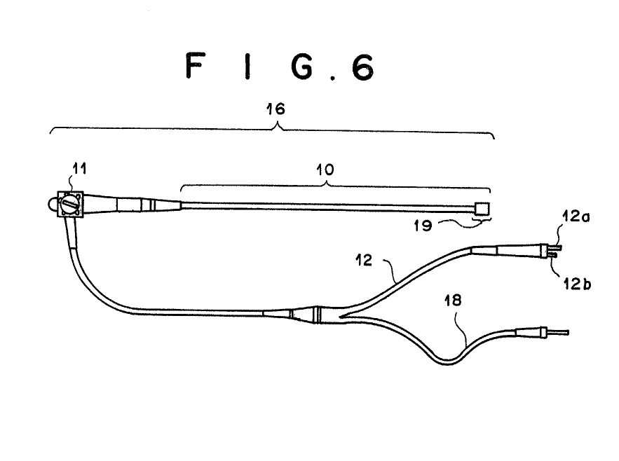

Still further, the illuminating-light guide 12 and the combined excitation light/reference-light light guides 14 and 15 can all be combined in a combined illuminating-light/excitation light/reference-light light guide 18, as shown in FIG. 6.

In addition, although according to the current embodiment, a configuration in which a reflectance image and a composite-image are displayed on a single monitor has been adopted, a configuration in which a separate monitor is provided for displaying each type of image, respectively, can also be adopted. Further, although according to the present embodiment a method of switching between the displaying of a composite-image and the displaying of a reflectance image by depressing a footswitch has been adopted, the switching can be performed automatically temporally by the control unit. Still further, the composite-image and the reflectance image can be superposed on one another and displayed.

Further, the excitation light/reference-light unit occurring in the current embodiment can be provided as a separate excitation light unit and a separate reference-light unit. Also, as to the excitation light source, any light source emitting light having a wavelength in the 400-420 nm wavelength range can be selected.

According to the fluorescent endoscope apparatus of the present invention, an image obtaining means 30, an illuminating-light unit 20, and an excitation light/reference-light unit 40 are provided mounted adjacently on a rack 100 located at a position remote from the scope means 16. The image obtaining means 30, the illuminating-light unit 20, and the excitation light/reference-light unit 40 are each connected to the scope means 16 by an image guide 13, an illuminating-light guide 12, and a set of combined excitation light/reference-light light guides 14 and 15, respectively, and because a configuration wherein the load of each unit is not brought to bear on the scope means 16 has been adopted, the operability of the scope means 16 can be improved. Further, because it becomes unnecessary to provide the support member and counterweight required to support a conventional scope means, the size of the apparatus can be made compact.

Further, the length of the illuminating-light guide 12 determines the range in which the scope means 16 can be moved, and because the length of the image guide 13 and the combined excitation light/reference-light light guides 14 and 15 have been made longer than the length of the illuminating-light guide 12, only the illuminating-light guide 12 limits the range of operability of the scope means 16, and because the combined excitation light/reference-light light guides 14 and 15 do not limit the range of operability of the scope means 16, the operability of the scope means 16 can be improved.

Still further, because the illuminating-light unit 20, and the excitation light/reference-light unit 40 are provided mounted adjacently within the rack 100, it is no longer necessary to make the image guide 13 and the combined excitation light/reference-light light guides 14 and 15 of a long length, whereby the operability is improved and the size of the apparatus can be made compact.

Additionally, although according to the current embodiment a configuration in which a reference-light image formed of the reflected-light reflected from a target subject upon the irradiation thereof by a reference-light has been employed, the current embodiment is not limited thereto; a configuration in which a reference-light image is not employed, and only a fluorescent-light image is employed can be adopted.

What is claimed is:

1. A fluorescent endoscope apparatus comprising: a
an illuminating-light emitting means for emitting an illuminating-light,
an excitation light emitting means for emitting an excitation light,
a reference-light emitting means for emitting a reference-light,
an illuminating-light guiding means for guiding the illuminating-light emitted from the illuminating-light emitting means to the target subject and projecting said illuminating-light onto the target subject,
an excitation light guiding means for guiding the excitation light emitted from the excitation light emitting means to the target subject and projecting said excitation light onto the target subject,
a reference-light guiding means for guiding the reference-light emitted from the reference-light emitting means to the target subject and projecting said illuminating-light onto the target subject,
a light guiding means for guiding a reflectance image formed of the reflected-light reflected from the target subject upon the irradiation thereof by the illuminating-light, a fluorescent-light image formed of the fluorescent light emitted from the target subject upon the irradiation thereof by the excitation light, and a reference-light image formed of the reflected-light reflected from the target subject upon the irradiation thereof by the reference-light,
an image obtaining means for obtaining a reflectance image, a fluorescent-light image, and a reference-light image guided thereto by the light guiding means, and
a scope means to be inserted into the body of a patient and which has disposed in the interior thereof the illuminating-light guiding means, the excitation light guiding means, the reference-light guiding means, and the light guiding means, wherein
said image obtaining means is provided at a location remote from the scope means, and
said image obtaining means and the light guiding means disposed within the scope means are optically connected by an image obtaining connecting means of a predetermined length.

2. A fluorescent endoscope apparatus as defined in claim 1, wherein
at least one from among the illuminating-light emitting means, the excitation light emitting means, and the reference-light emitting means is provided at a position remote from the scope means, and
said at least one light emitting means provided at a position remote from the scope means and the light guiding means corresponding to said at least one light emitting means are optically connected by one or more connecting means of a predetermined length.

3. A fluorescent endoscope apparatus as defined in claim 2, wherein
one connecting means from among said one or more connecting means and the image obtaining connecting means is of a length determining the distance between the light emitting means or the image obtaining means corresponding to said one connecting means and the scope means, and
the connecting means other than said one connecting means having said length determining said distance can be longer in length than said one connecting means whose length determines said distance.

4. A fluorescent endoscope apparatus as defined in either of claims 2 or 3, wherein
at least two from among the illuminating-light emitting means, the excitation light emitting means, the reference-light emitting means, and the image obtaining means provided at a position remote from the scope means are disposed adjacent to one another.

5. A fluorescent endoscope apparatus comprising: a
an illuminating-light emitting means for emitting an illuminating-light,
an excitation light emitting means for emitting an excitation light,
an illuminating-light guiding means for guiding the illuminating-light emitted from the illuminating-light emitting means to the target subject and projecting said illuminating-light onto the target subject,
an excitation light guiding means for guiding the excitation light emitted from the excitation light emitting means to the target subject and projecting said excitation light onto the target subject,
a light guiding means for guiding a reflectance image formed of the reflected-light reflected from the target subject upon the irradiation thereof by the illuminating-light, and a fluorescent-light image formed of the fluorescent light emitted from the target subject upon the irradiation thereof by the excitation light,
an image obtaining means for obtaining a reflectance image, and a fluorescent-light image there to by the light guiding means, and
a scope means to be inserted into the body of a patient and which has disposed in the interior thereof the illuminating-light guiding means, the excitation light guiding means, and the light guiding means, wherein
said image obtaining means is provide data location remote from the scope means, and
said image obtaining means and the light guiding means disposed within the scope means are optically connected by an image obtaining connecting means of a predetermined length.

6. A fluorescent endoscope apparatus as defined in claim 5, wherein
at least one from among the illuminating-light emitting means, and the excitation light emitting means, is provided at a position remote from the scope means, and
said at least one light emitting means provided at a position remote from the scope means and the light guiding means corresponding to said at least one light emitting means are optically connected by one or more connecting means of a predetermined length.

7. A fluorescent endoscope apparatus as defined in claim 6, wherein
one connecting means from among said one or more connecting means and the image obtaining connecting means is of a length determining the distance between the light emitting means or the image obtaining means corresponding to said one connecting means and the scope means, and
the connecting means other than said one connecting means having said length determining said distance can be longer in length than said one connecting means whose length determines said distance.

8. A fluorescent endoscope apparatus as defined in claim 6 or 7, wherein
at least two from among the illuminating-light emitting means, the excitation light emitting means, and the image obtaining means provided at a position remote from the scope means are disposed adjacent to one another.

9. A fluorescent endoscope apparatus as defined in any of the claims 1, 2, 3, 5, 6, or 7, wherein
the excitation light source is a GaN semiconductor laser which emits excitation light having a wavelength in the 400-420 nm wavelength range.
